(19) United States
(12) Patent Application Publication
(10) Pub. No.: US 2026/0338690 A1
(43) Pub. Date: Oct. 29, 2026

(54) METHOD AND APPARATUS FOR MODULATING SIGNALS OF LUMINESCENT POLYMER/DYE FORMULATIONS USING LIGHT SCATTERING PARTICLES

(71) Applicants: The General Hospital Corporation, Boston, MA (US); Solventum Intellectual Properties Company, Maplewood, MN (US)

(72) Inventors: Matthias Müller, Boston, MA (US); Conor L. Evans, Boston, MA (US); Emmanouil Rousakis, Charlestown, MA (US); Juan Pedro Cascales Sandoval, Boston, MA (US); Jason Bjork, Cottage Grove, MN (US); Dawn Muyres, Stillwater, MN (US); Elias Wilken-Resman, Minneapolis, MN (US); Döne Demirgöz, St. Paul, MN (US); Gabriela Apiou, Medford, MA (US)

(21) Appl. No.: 19/111,756

(22) PCT Filed: Sep. 20, 2023
(86) PCT No.: PCT/US2023/074688

§ 371 Date: Mar. 13, 2025

Related U.S. Application Data
(60) Provisional application No. 63/409,277, filed on Sep. 23, 2022.

(51) Int. Cl. G01N 21/64

(52) U.S. Cl. CPC G01N 21/64; G01N 2201/062; G01N 2201/0634

(57) ABSTRACT

A sensing system and method for sensing an analyte. The sensing system includes; a sensing material having one or more layers, the sensing material including; a light emitting material and a plurality of signal enhancing particles disposed within a polymer matrix. The sensing method includes: providing a sensing material including one or more layers, the sensing material including a light emitting material and a plurality of signal enhancing particles disposed within a polymer matrix, the light emitting material being sensitive to an analyte; exposing the sensing system to the analyte; and measuring a change in a light emission from the light emitting material based on exposing the sensing system to the analyte.

### CROSS-REFERENCE TO RELATED APPLICATIONS

The present application is based on and claims priority from U.S. Patent Application No. 63/409,277, filed on Sep. 23, 2022, the entire disclosure of which is incorporated herein by reference.

### FIELD OF THE DISCLOSURE

The disclosed technology is generally directed to enhancing luminescence intensity. More particularly the technology is directed to oxygen-sensing bandages with enhanced optical properties.

### BACKGROUND OF THE DISCLOSURE

Oxygen is a key metabolite in cellular respiration and is required for adenosine triphosphate (ATP) synthesis. As insufficient oxygen delivery may result in irreversible tissue damage, early detection of compromised tissue oxygenation is of great importance. There has been a growing interest in developing “smart” wearable, oxygen-sensing devices for continuous health monitoring in both clinical fields and home settings. For example, tissue oxygenation monitoring is of particular importance during the wound healing process, as reduced oxygenation is a contributing factor in both acute and chronic wound pathogenesis. Therefore, the development of oxygen-sensing materials is of great interest for both health monitoring and clinical diagnostics. Towards this end, oxygen-interactive luminescent organic dyes have been developed for a variety of imaging and monitoring applications. The field of oxygen measurement via phosphorescence quenching is a particularly important and growing field. Despite significant progress, oxygen sensitive phosphors incorporated into wearable devices are hindered by low emission intensity and require the use of expensive optics and detectors.

### BRIEF SUMMARY OF THE DISCLOSURE

Accordingly, embodiments of the technology disclosed herein address one or more of the issues identified above by incorporating light scattering or reflective particles into a polymer-luminescent material matrix to improve emission intensity by redirecting photons, which are initially emitted by the luminescent material in directions away from the detector, back towards the detector. Notably, this technology and method may be used to improve luminescence intensity in applications outside of oxygen sensing and may act as both an enhancer of luminescence intensity as well as indicator of changing conditions in the underlying sample (e.g. a wound) via material changes in optical scatterer properties. Furthermore, this approach is broadly applicable and is not limited to a particular luminescent material chemistry or mechanism of photoemission.

Thus, disclosed herein are sensing materials and methods of using the same. Embodiments of the sensing material include a light emitting material responsive to an analyte of interest (also referred to simply as an “analyte”), a plurality of signal enhancing particles, and one or more polymer matrices in which the light emitting material and signal enhancing/scattering particles are disposed. In certain embodiments, the light emitting material that is responsive to an analyte of interest includes a light emitting material that undergoes at least one of a change in intensity or a change of spectral properties based on exposure to the analyte of interest. In one embodiment, the analyte of interest includes oxygen and the light emitting material includes a metalloporphyrin. The plurality of signal enhancing particles (also referred to as “scatterers” or “scattering particles”) include light scattering or reflective particles. In some embodiments, the signal enhancing particles include at least one of glass (e.g. solid or hollow particles such as spheres), titanium dioxide, zinc, barium sulfate, diamond, polystyrene, or polypropylene. In one embodiment the material is constructed in two or more independently tunable layers. In one embodiment, a first tunable layer includes the light emitting material responsive to an analyte of interest along with a polymer, and a second tunable layer includes a plurality of signal enhancing particles. In one embodiment, the second tunable layer including a plurality of signal enhancing particles is positioned between, and in contact with, both of a sample of interest (such as a wound such as a skin lesion) and the first tunable layer including the light emitting material responsive to analyte of interest. There may also be an optional adhesive layer, between the scattering layer and the sample, however in some embodiments the scattering layer itself may include an adhesive, instead of or in addition to the separate adhesive layer. Similarly, in some embodiments the single layer of material may also include adhesive or there may be a separate adhesive layer between the single layer of material and the sample. In some embodiments the polymer matrix may be or include an adhesive.

In some embodiments, the light emitting material may or may not be responsive to the analyte of interest and instead the scattering or reflective particles may be responsive to the analyte of interest. The scattering or reflective particles may undergo an interaction with underlying sample conditions, such as absorption of an analyte or release of a drug, that may change the optical scattering or reflective properties of the signal enhancing scattering or reflective particles. In some embodiments, the interaction may cause changes in at least one of index of refraction, absorptive properties, scattering angle, size, shape, orientation, or number of scatterers.

Also disclosed herein is a system for measuring an analyte of interest including the sensing material described above provided in conjunction with an excitation source and a detector. In some embodiments, the excitation source is a flash (e.g. a flash source such as an electronic source) and the detector is a camera. In another embodiment, the excitation source is a laser or an LED and the detector is a photodiode. In one embodiment, the system for measuring the analyte of interest includes exposing the sensing material to conditions where the analyte may be present and, following illumination, monitoring at least one of the luminescence intensity, scattering angle, or luminescence lifetime of the light emitting material responsive to the analyte of interest.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1A demonstrates a construction wherein only a semitransparent luminescent layer is present with no scattering or backscattering layer, demonstrating how photons are lost when they are emitted in directions away from photodetectors.

FIG. 1B shows a construction according to the present disclosure wherein the luminescent layer and scattering layer are separate layers positioned so that the luminescent layer is between the scattering layer and the excitation source and photodetectors. In one construction, the matrix of the scattering layer and the matrix of the luminescent layer are made from different materials.

FIG. 1C shows a construction according to the present disclosure wherein the luminescent layer and scattering layer are one layer. In one construction, the matrix of the scattering layer and the matrix of the luminescent layer are the same material.

FIG. 2A shows a construction according to the present disclosure wherein photons emitted by an excitation source impinge upon the disclosed sensing material, which in this construction includes separate semi-transparent luminescent and scattering layers, where the luminescent layer includes a light emitting material (which may or may not be responsive to an analyte of interest). The interaction of the scatterers with the analyte of interest, which may include absorption of an analyte or release of a drug, may cause an increase or decrease in one or more wavelengths of light emitted and scattered by the scattering layer due to changes to the scatterers such as a change in index of refraction.

FIG. 2B shows a construction according to the present disclosure wherein photons emitted by an excitation source impinge upon the disclosed sensing material, which includes a semi-transparent luminescent layer and a scattering layer. The interaction of the matrix surrounding the scatterers with an analyte of interest (absorption of an analyte or release of a drug) can cause a change to the material surrounding the scatterers (e.g. a change in index of refraction or absorptive properties) which in turn can cause an increase (or decrease) in one or more wavelengths of light scattered by the scattering particles.

FIG. 3 shows a construction according to the present disclosure wherein photons emitted by an excitation source impinge upon the disclosed sensing material, which includes a semi-transparent luminescent layer and a scattering layer. The interaction of the scattering layer with the analyte of interest (e.g. absorption of an analyte or release of a drug) can cause a change in scattering angle of the photons scattered by the scattering layer. The interaction may cause a change in the scattering layer, such as a change, increase, or decrease in one or more scattering angles of one or more wavelengths of scattered light.

FIG. 4 shows a construction according to the present disclosure wherein photons emitted by an excitation source impinge upon the disclosed sensing material, which includes a semi-transparent luminescent layer and a scattering layer. The interaction of the scattering layer with the analyte of interest (e.g. absorption of an analyte or release of a drug) can cause a change in the scattering layer, such as a change in the size, shape, or orientation of the scatterers. In one instance, the change in scatterer size, shape, or orientation results in an increase or decrease in one or more scattering angles of one or more wavelengths of scattered light or an increase or decrease in intensity of one or more wavelengths of scattered photons.

FIG. 5 shows a construction according to the present disclosure wherein photons emitted by an excitation source impinge upon the disclosed sensing material, which includes a semi-transparent luminescent layer and a scattering layer. The interaction of the scattering layer with the analyte of interest (absorption of an analyte or release of a drug) can cause a change in change in scatterer number, such as an increase or decrease in the number of scatterers. In one instance, the change in scatterer number results in an increase or decrease of in one or more scattering angles of one or more wavelengths of scattered light or an increase or decrease in intensity of one or more wavelengths of scattered photons.

FIG. 6 shows a construction according to the present disclosure wherein Mie scattering is leveraged by incorporating Mie scatterers into a scattering layer. Mie scatterers scatter photons directionally forward and backward relative to the direction of the incident photon. In one construction, the Mie scattering layer is positioned between the excitation source and an additional reflective layer that is configured to reflect the forward scattered light towards the photodetector.

FIG. 7 shows a construction according to the present disclosure wherein distributed rod-shaped particles serve as light scatterers in addition to scale bars for automating camera image processing and analysis through incorporation of techniques, including machine learning, to compensate for variables such as camera distance and orientation.

FIG. 8 shows luminescence intensity and luminescence decay time of air-exposed and N₂-exposed samples of isooctyl acrylate (IOA)-based adhesive embedded with cyclohexenyl pivaloyl Pt-porphyrin. First, the cyclohexenyl pivaloyl Pt-porphyrin was dissolved in ethyl acetate to create a stock solution. This solution was added to the IOA adhesive. The mixture was mixed for 24 h on a benchtop roller. The mixture was subsequently coated onto 3M Medical Tape 1525L using a spin coater with sufficient volume of dye solution to flood a 50 mm diameter circular sample. Panel A shows a control sample of only the matrix polymer and the cyclohexenyl pivaloyl Pt-porphyrin on the substrate. Panel B shows the cyclohexenyl pivaloyl Pt-porphyrin in IOA-based adhesive embedded with hollow glass spheres. Panel C shows cyclohexenyl pivaloyl Pi-porphyrin in in IOA-based adhesive embedded with TiO₂ particles. Emission intensity was assessed using a Nikon D3400 DSLR camera equipped with 60 mm lens and two bilaterally mounted Vivitar flash units. A blue/UV bandpass filters ( 385/70 nm, Chroma Technologies) was mounted on the lens and red-green dual bandpass filters ( 525/30 nm+ 650/40 nm) were mounted on the flash units. The distance between the lens and the oxygen-sensing film was ~35 cm. Exposure time and aperture number were 0.01 s and f/11, respectively. ISO was set to 100. Afterwards, images were separated in red, green, and blue channels to assess the red and green signal intensity.

### DETAILED DESCRIPTION OF THE DISCLOSURE

Luminescent organic dyes have been developed for a variety of imaging and monitoring applications. The field of oxygen measurement via phosphorescence quenching is a particularly important and growing field. Oxygen sensing through phosphorescence quenching dates back to the 1930s and has been developed into an established technique. Measuring oxygen concentration through phosphorescence quenching has ample of benefits compared to traditional techniques. First, this approach provides a direct measurement of tissue oxygenation, while other techniques only provide indirect measurements (e.g. blood oxygenation). Pulse oximetry, a common tool to measure blood oxygen saturation, relies on pulsatile blood flow, is affected by varying skin color and competing molecules, and is prone to error if the subject is not still. Current transcutaneous oxygen monitoring (TCOM) tools as well as blood gas analysis require bulky devices and trained personnel. In contrast, oxygen sensors based on luminescence quenching can be made small and are easy to operate.

The method of oxygen measurement through luminescence quenching relies on measuring the change in luminescence intensity or lifetime of certain molecules when exposed to oxygen. The collision of molecular oxygen with the excited-state probe results in energy transfer from the probe to the oxygen molecule via triplet-triplet-interaction. This energy transfer causes a decrease in intensity and lifetime; in this way both lifetime and intensity are inversely proportionally to oxygen concentration. The limit of detection of the probe depends on the lifetime of the excited state of the oxygen-sensing material.

These oxygen-sensing molecules can be incorporated into a polymer matrix for stabilization and easy handling. Since detection sensitivity can be related to the lifetime of the of the excited state of the oxygen-sensitive molecule, concentrations of the oxygen-sensitive molecule should be kept as low as possible to avoid aggregation. Aggregation of the probe can result in energy transfer between the molecules with a subsequently reduction in luminescence lifetime and limit of detection. However, reducing the concentration of the probe also results in a decrease in emission intensity and signal strength which in return requires the use of expensive optics and detectors.

We have found that, depending on the geometry of excitation and detection, by adding scattering or reflective particles, particularly titanium dioxide particles and glass beads, to the polymer/dye formulations, the detected emission intensity and thus measured signal strength could be increased by several factors (e.g. see FIG. 8 and discussion below). These scattering/reflective particles can include metals, metal oxides, polymer beads, glass beads, similar materials, or a combination thereof. The scattering of light depends on both particle size and the wavelength of the light. When light is scattered by particles smaller than a tenth of the wavelength, light scattering is dominated by the Rayleigh scattering mechanism. When the particle size is the same size or larger than wavelength of the light, Mie scattering instead dominates. Mie scattering can be stronger than Rayleigh scattering and can also be directional. This implies that the selection of particle size could be used to optimize both the directionality and amount of scattered light to modulate or improve the detected light signal levels.

In one embodiment of this disclosure, the light scattering material may be used to indicate features of the material or of the tissue it is in contact with. For example, the scattering property could be modulated by the amount of a drug released from a film or bandage, such that the measured scattered light, in combination with oxygen sensing, could indicate drug release quantitatively or semi-quantitatively. The scattering material can be made to change in response to tissue properties, such as water content, amount of exudate/transudate released, or the presence of bacteria or specific species of bacteria. Scattering changes could be used to detect or distinguish diseased vs healthy tissue, such as for example, determining the margins of a wound beneath the bandage. The scattering material can also be set so that it could act as a “timer” or indicator of the film or bandage lifetime or wear time, such that a change in scattering could act as a personalized indicator that a bandage change or replacement is necessary, for example. As the size of the scatterer influences the spectrum of light scattered, such changes as listed above could also be encoded in changes to the light scattering spectrum and read out by measurement of the spectrum or chosen spectral regions. These changes in scattering can arise from changes or modifications to individual scattering elements, such as scatter size and shape, as well as the aggregate status of the scatterers, such as proximity.

It is also worth noting that such changes in these scatters could also be set so that an intervention could change or tune the scattering properties. For example, the scatters could be exposed to light which could be used to increase or decrease the scattering efficiency; such an approach could be used for manual or automatic “tuning” of the material property to maximize function of films and bandages such as signal to noise ratio or total signal intensity. Chemical treatment of the scatters could also be used to modulate scattering properties, where chemical treatments could be compounds typically used in clinical care, such as but not limited to isopropanol or iodide antiseptics. Chemical treatments could also be specifically selected or compounded formulations or solutions that react with or alter the scatterers to achieve similar changes.

The approach used in current devices to increase signal intensity has been the application of multi-layer architecture. In this multi-layer approach, the luminescent formulation is usually cast onto a reflective substrate, e.g. polytetrafluoroethylene. A distinguishing feature of the present innovation is the combination of the luminescent formulation and a reflective or scattering layer by using reflective or scattering particles. These scattering or reflecting particles may be dispersed within the formulation (or in a polymer matrix which includes the formulation) resulting in a single layer that contains both components, including the luminescent formulation as well as the scattering material. Combining the luminescent layer with the reflective layer into a single layer would allow for a smaller and simpler architecture as well as the reduction of materials and costs. Such an approach may also for more facile manufacturing of materials. Nevertheless, in some embodiments the luminescent formulation may be present in a separate layer from the scattering or reflective elements. Further, the scattering or reflective elements can also be created such that their scattering/reflective properties are tunable to both internal or external stimuli, including the presence of water or bacteria, or such as the detection of healthy tissue from diseased tissue.

In various embodiments, the procedures of the present disclosure can find application not only in oxygen-sensing devices, such as wearable devices or paint-on bandages but also in any type of application that requires luminescent thin-films that suffer from a low emission intensity.

In general, the system is designed such that a sensing material is placed close to a sample of interest (e.g. a film or bandage which includes the sensing material might be placed adjacent to a wound such as a skin lesion) and properties of the sample are monitored using the sensing material. The sample may release a material (e.g. oxygen) that is monitored by the sensing material, or the sensing material may release a material into the sample (e.g. a drug) whose release levels or remaining amounts are monitored by the sensing material. In some embodiments, the sensing material may include a light emitting material responsive to an analyte of interest, where the properties of the light emitting material are affected by the presence or absence of the analyte of interest, as discussed further below. In other embodiments, the light emitting material may or may not be responsive to an analyte of interest and the sensitivity of the sensing material to the analyte of interest may be conferred by other components in the sensing material such as scattering particles and/or a matrix which contains the scattering particles, as discussed further below.

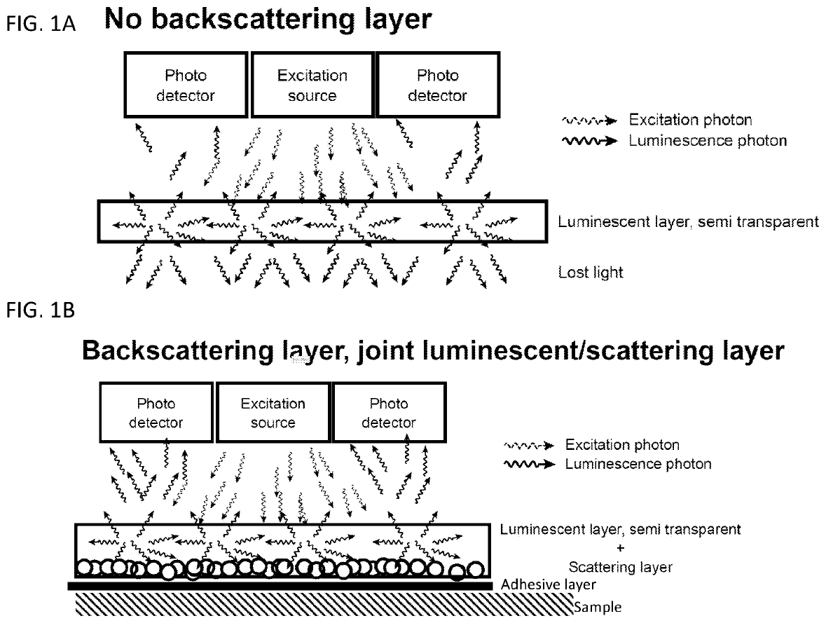

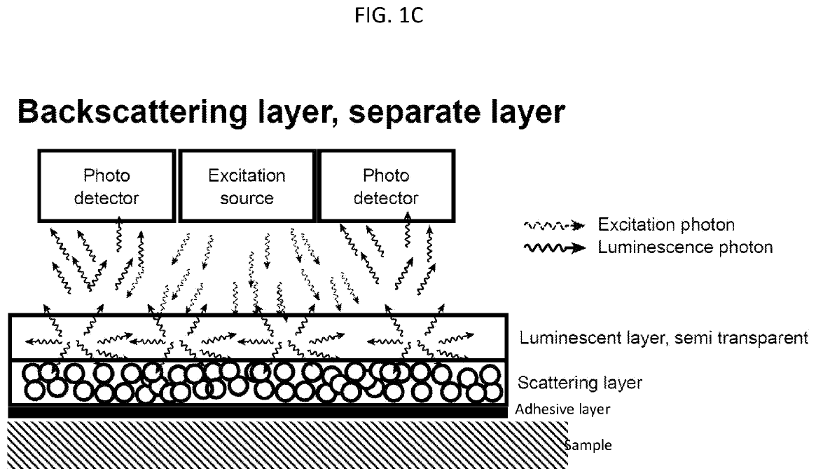

In the absence of scattering particles, a certain amount of the light from the luminescent layer is not detected because it is emitted in directions away from the photo detector (FIG. 1A). Embodiments of the present disclosure are provided which address this problem by redirecting at least a portion of the light that is emitted in a direction away from the detector toward the detector to improve the overall signal that is obtained. The light may be redirected by reflection and/or scattering of emitted photons by the scattering particles towards the detectors. FIG. 1B shows an embodiment in which the scattering particles are disposed within a semi-transparent luminescent layer along with a light emitting material, such that the sensing material is made of a single layer of material; while the scattering particles are shown in the embodiment of FIG. 1B as being on one side of the layer, in other embodiments the scattering particles may be distributed throughout the layer. As shown in FIGS. 1B and 1C, the sensing material may be placed on or near a sample such as a wound (e.g. a skin lesion) on a subject (e.g. a human or animal subject). FIG. 1C shows another embodiment which includes separate luminescent and scattering layers. Various examples disclosed herein which are presented as having a single layer may also be implemented using two or more layers, with components distributed between the layers in different proportions. Likewise, examples disclosed herein which are presented as having two or more layers may also be implemented using a single layer. Accordingly, both the single-layer and the multi-layer embodiments are encompassed with the scope of the present disclosure. In addition, while FIGS. 1B and 1C depict a sample against which the sensing material is placed, the sample has been omitted from the remaining images for the sake of clarity. FIG. 1C includes an optional adhesive layer, between the scattering layer and the sample, however in some embodiments the scattering layer itself may include an adhesive, instead of or in addition to the separate adhesive layer. Similarly, in some embodiments such as FIG. 1B the single layer of material may also include adhesive or there may be a separate adhesive layer between the single layer of material and the sample. In some embodiments the polymer matrix may be or include an adhesive.

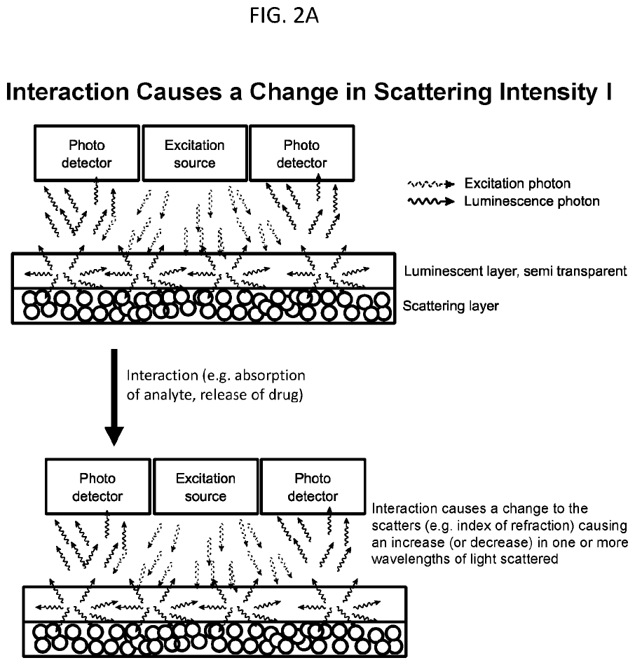

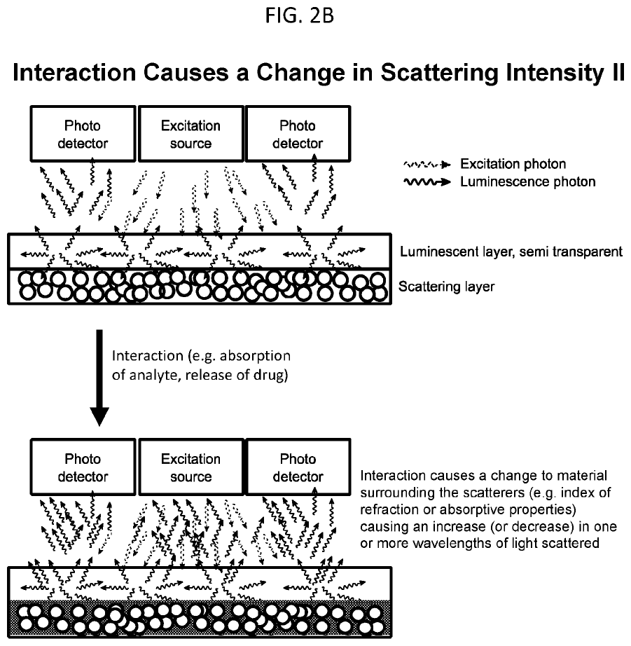

FIG. 2A shows an embodiment according to the present disclosure wherein photons emitted by an excitation source impinge upon the disclosed sensing material, which in this embodiment includes separate semi-transparent luminescent and scattering layers, where the luminescent layer includes a light emitting material (which may or may not be responsive to an analyte of interest). The interaction of the scatterers with the analyte of interest, which may include absorption of an analyte or release of a drug, may cause an increase or decrease in one or more wavelengths of light emitted and scattered by the scattering layer due to changes to the scatterers such as a change in index of refraction.

FIG. 2B shows an embodiment according to the present disclosure wherein photons emitted by an excitation source impinge upon the disclosed sensing material, which includes a semi-transparent luminescent layer and a scattering layer. The interaction of the matrix surrounding the scatterers with an analyte of interest (absorption of an analyte or release of a drug) can cause a change to the material surrounding the scatterers (e.g. a change in index of refraction or absorptive properties) which in turn can cause an increase (or decrease) in one or more wavelengths of light scattered by the scattering particles. For the embodiments of FIGS. 2A and 2B, changes in the optical properties of the scatterers or the surrounding matrix, or both, due to interaction with an analyte of interest can lead to changes in the light-scattering properties of the scatterers that can be detected by the photo detector. In some embodiments, materials that undergo changes in refractive index when absorbing water may be used as the scattering particles, including hydrogels or swellable polymer block co-polymers. In one embodiment, with increasing water content, the refractive index of hydrogels decreases. In other embodiments, the scattering particles may include PVA, Polyacrylic acid, PDMS, polystyrene, or polystyrene block-co-polymers. In other embodiments, the matrix may include PDMS, PVA, PEG, alginate, colloidal hydrogel, collagen, or hyaluronan. In various embodiments any combination of scattering particle materials may be used with any combination of matrix materials, provided that there is a sufficient difference in refractive index between the scattering particle materials and the matrix materials. In one embodiment the scattering particles may be no lower than 10 nanometers and no lower than 100 micrometers.

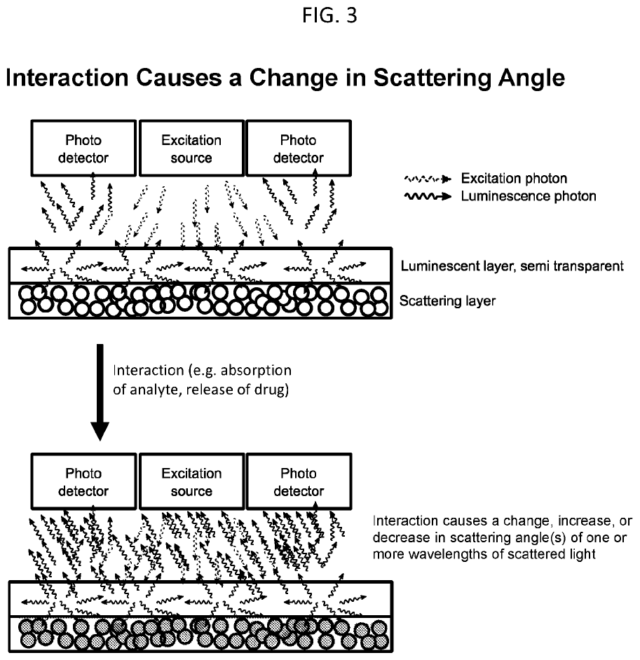

FIG. 3 shows an embodiment according to the present disclosure wherein photons emitted by an excitation source impinge upon the disclosed sensing material, which includes a semi-transparent luminescent layer and a scattering layer. The interaction of the scattering layer with the analyte of interest (e.g. absorption of an analyte or release of a drug) can cause a change in scattering angle of the photons scattered by the scattering layer. The interaction may cause a change in the scattering layer (e.g. a change in the scattering particles and/or the matrix), such as a change, increase, or decrease in one or more scattering angles of one or more wavelengths of scattered light. In one embodiment the angular scattering pattern is altered by changes induced in scattering particle aspect ratio. In some embodiments, the scattering particle shape may change from or to rods, discs, or spheres. In one embodiment, changes in scattering angle may be monitored, e.g. by using filters which only transmit a narrow solid angle cone of light. This way, if the scattering angle deviates from the initial conditions by a certain amount, the angle filter would cause a change in the measured intensity. In other embodiments, the scattering particles may include PVA, PDMS, polystyrene, polystyrene block-co-polymers. In other embodiments, the matrix may include PVA, PDMS, polystyrene, polystyrene block-co-polymers. In various embodiments any combination of scattering particle materials may be used with any combination of matrix materials, provided that there is a sufficient difference in refractive index between the scattering particle materials and the matrix materials. In one embodiment the scattering particles may be no lower than 10 nanometers and no lower than 100 micrometers.

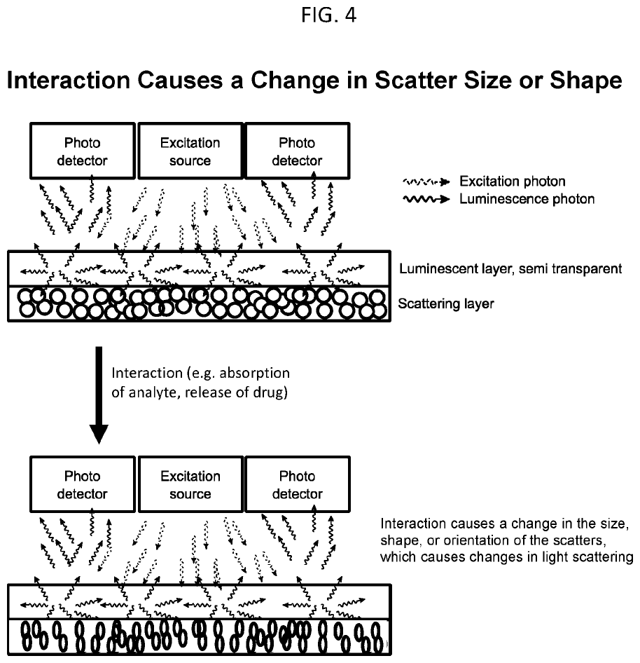

FIG. 4 shows an embodiment according to the present disclosure wherein photons emitted by an excitation source impinge upon the disclosed sensing material, which includes a semi-transparent luminescent layer and a scattering layer. The interaction of the scattering layer with the analyte of interest (e.g. absorption of an analyte or release of a drug) can cause a change in the scattering layer, such as a change in the size, shape, or orientation of the scatterers. In one embodiment, the change in scatterer size, shape, or orientation results in an increase or decrease in one or more scattering angles of one or more wavelengths of scattered light or an increase or decrease in intensity of one or more wavelengths of scattered photons. In one embodiment, interaction with an analyte may cause changes in the size and/or aspect ratio of the particles, as well as their orientation. In one embodiment, scattering particles may include rods or stacked disks that alter their shape (e.g. size, aspect ratio) upon analyte interaction. In another embodiment, scattering particles can be enmeshed in a matrix that, upon interaction, escape the mesh. Particles that contain a drug (e.g. PS, PMMA, PDMS) can be either degraded or change in size upon release of drug. In other embodiments, the scattering particles may include PVA, PDMS, polystyrene, polystyrene block-co-polymers. In other embodiments, the matrix may include PVA, PDMS, polystyrene, polystyrene block-co-polymers. In various embodiments any combination of scattering particle materials may be used with any combination of matrix materials, provided that there is a sufficient difference in refractive index between the scattering particle materials and the matrix materials.

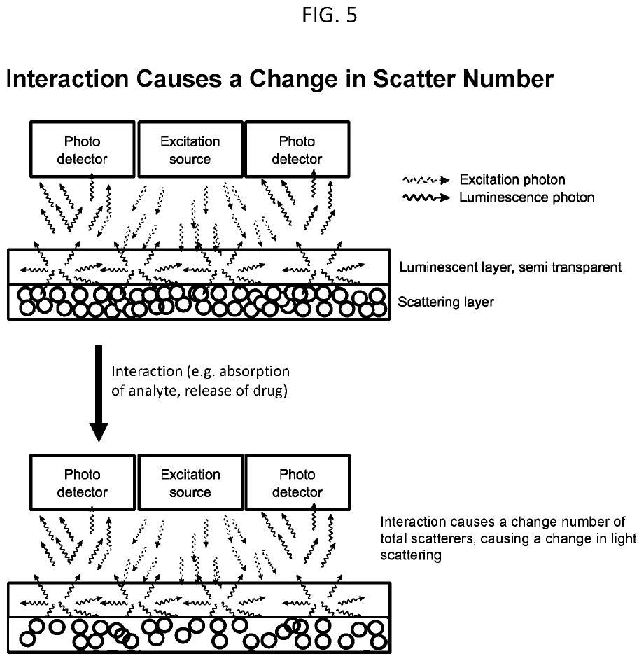

FIG. 5 shows an embodiment according to the present disclosure wherein photons emitted by an excitation source impinge upon the disclosed sensing material, which includes a semi-transparent luminescent laver and a scattering layer. The interaction of the scattering layer with the analyte of interest (absorption of an analyte or release of a drug) can cause a change in change in scatterer number, such as an increase or decrease in the number of scatterers. The number of scatterers may change due to an increase or decrease in aggregation of the scattering particles which is influenced by the presence of the analyte of interest; while the number of actual particles may not change, the changes in aggregation would lead to changes in how the particles (or clumps of particles) behave for the purpose of scattering, creating a higher or lower effective number of scatterers. In one instance, the change in scatterer number results in an increase or decrease of in one or more scattering angles of one or more wavelengths of scattered light or an increase or decrease in intensity of one or more wavelengths of scattered photons. In some embodiments, a change in scattering particle number may occur if particles dissolve or breakdown in the presence of an analyte. In this embodiment, only a fraction of the particles may need to either be interacting or interact for this effect. In another embodiment, scattering particles may be made of multiple scattering particles linked together and the analyte may cause the break of the linkage, and may result in a decrease in particle size but increase in particle number, changing light scattering properties. In some embodiments, the scattering particles may include PVA, PDMS, polystyrene, polystyrene block-co-polymers. In other embodiments, the matrix may include PVA, PDMS, polystyrene, polystyrene block-co-polymers. In various embodiments any combination of scattering particle materials may be used with any combination of matrix materials, provided that there is a sufficient difference in refractive index between the scattering particle materials and the matrix materials.

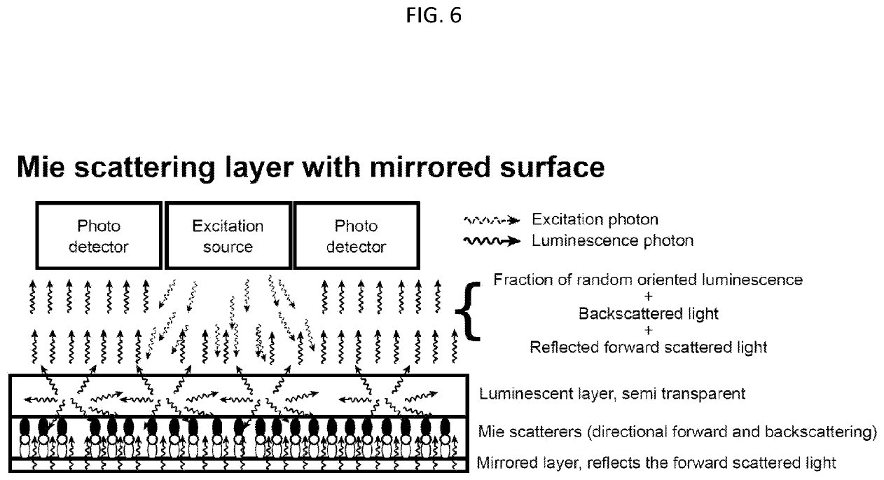

FIG. 6 shows an embodiment according to the present disclosure wherein Mie scattering is leveraged by incorporating Mie scatterers into a scattering layer, although in some embodiments the Mie scatters may be incorporated into the luminescent layer and do not necessarily have to be disposed in a separate layer. Mie scatterers scatter photons directionally forward and backward relative to the direction of the incident photon. In one particular embodiment, the Mie scattering layer is positioned between the excitation source and an additional mirror layer that is configured to reflect the forward scattered light towards the photodetector. In one embodiment, the matrix itself may change upon interaction with an analyte. For example, water absorption may change the surrounding index of refraction which may alter scattering intensity and angle. In some embodiments, scattering particles could be one or any combination of TiO₂, SiO₂, BaSO₄, ZnO, gold, silver, tin. In other embodiments, the scattering particles may include PVA, PDMS, polystyrene, polystyrene block-co-polymers. In other embodiments, the matrix may include PVA, PDMS, polystyrene, polystyrene block-co-polymers. In various embodiments any combination of scattering particle materials may be used with any combination of matrix materials, provided that there is a sufficient difference in refractive index between the scattering particle materials and the matrix materials. One skilled in the art will recognize that Mie scattering is a function of particle size and predominantly occurs when the wavelength of the incident light is within a couple order of magnitude of the incident light. In some embodiments, the wavelength of incident light is no smaller than 0.1 nm and no longer than 1000 micrometers.

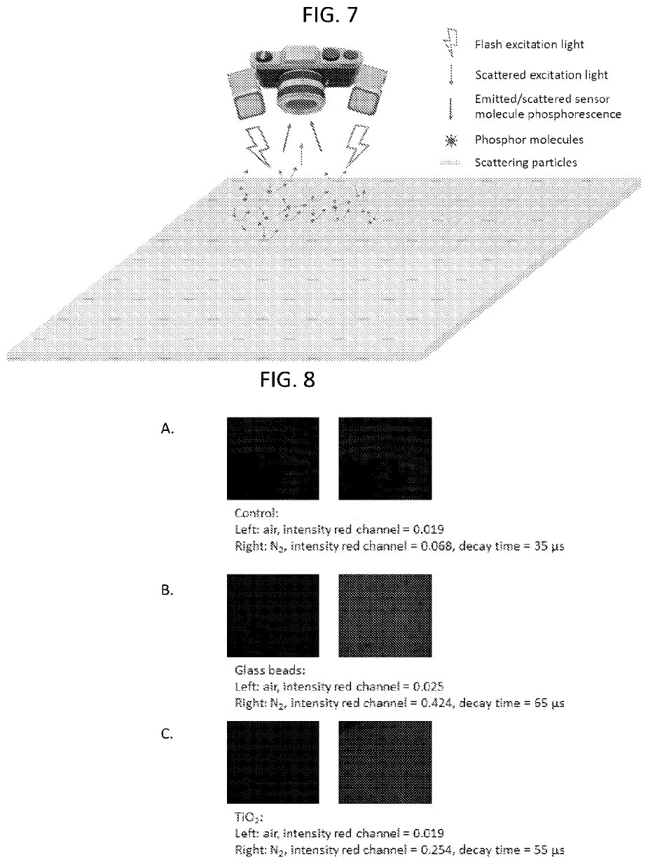

FIG. 7 shows a construction according to the present disclosure wherein distributed particles can serve as light scatterers. In various embodiments, the distributed particles may be homogenously sized or may vary in size. In various embodiments, the rod-shaped particles comprised of materials including metals such as silver and gold may range in size from 10 nm to 100 μm, with all of the particles within a particular sensing material sample being substantially uniform in length, e.g. within at least 10% of the nominal length. It should be noted that this design could include separate built-in scale bars that would facilitate automation of camera image processing and analysis which could further be aided by incorporation of techniques, including machine learning, to compensate for variables such as camera distance and orientation.

FIG. 8 provides proof-of-concept results for the disclosed procedures. FIG. 8 shows luminescence intensity and luminescence decay time of air-exposed and N₂-exposed samples of isooctyl acrylate (IOA)-based adhesive embedded with cyclohexenyl pivaloyl Pt-porphyrin. First, the cyclohexenyl pivaloyl Pt-porphyrin was dissolved in ethyl acetate to create a stock solution. This solution was added to the IOA adhesive. The mixture was mixed for 24 h on a benchtop roller. The mixture was subsequently coated onto 3M Medical Tape 1525L using a spin coater with sufficient volume of dye solution to flood a 50 mm diameter circular sample. Panel A shows a control sample of only the matrix polymer and the cyclohexenyl pivaloyl Pt-porphyrin on the substrate. Panel B shows the cyclohexenyl pivaloyl Pt-porphyrin in IOA-based adhesive embedded with hollow glass spheres. Panel C shows cyclohexenyl pivaloyl Pt-porphyrin in in IOA-based adhesive embedded with TiO₂ particles.

Emission intensity was assessed using a Nikon D3400 DSLR camera equipped with 60 mm lens and two bilaterally mounted Vivitar flash units. A blue/UV bandpass filters ( 385/70 nm, Chroma Technologies) was mounted on the lens and red-green dual bandpass filters ( 525/30 nm+ 650/40 nm) were mounted on the flash units. The distance between the lens and the oxygen-sensing film was =35 cm. Exposure time and aperture number were 0.01 s and f/11, respectively. ISO was set to 100. Afterwards, images were separated in red, green, and blue channels to assess the red and green signal intensity.

Accordingly, disclosed herein are sensing materials and methods of using the same. The materials include a light emitting material responsive to an analyte of interest, a plurality of signal enhancing particles, and a polymer matrix. The light emitting material responsive to analyte of interest includes a light emitting material that undergoes at least one of a change in intensity or a change of spectral properties based on exposure to the analyte of interest. Also disclosed herein is a system for measuring an analyte of interest comprising the sensing material described above which may further include an excitation source and a detector. In one embodiment, the excitation source and detector are generally orthogonal to the sensing material (e.g. see FIGS. 1-6). In one embodiment, a sample may be positioned in close proximity to the sensing material and the sensing material may be positioned between the sample and the excitation source and detector. In some embodiments, the sample may be a wound or healthy skin.

Without being limited as to theory, the signal enhancing particles may enhance the intensity of the light emitting material responsive to an analyte of interest by redirecting photons that are emitted in directions away from the detector back toward the detector through reflection or scattering. Notably, the photons being scattered or reflected in various embodiments of the present disclosure originate from luminescence of a light emitting material. For example, in one embodiment the light emitting material may luminesce via electronic relaxations from triplet to singlet states, resulting in isotropically emitted photons. Some of the emitted photons will have trajectories traveling away from the detector and, without intervention, would not impinge upon the detector and would go undetected. The present disclosure teaches that these photons which would otherwise be lost may instead be scattered or reflected in the direction of the detector by the addition of scattering or reflective particles, thereby increasing the number of photons reaching the detector and thus increasing the overall efficiency of the system.

In some embodiments, the detected signal is an emission property or a change in emission property. In some embodiments, the emission property is luminescence intensity. In other embodiments, the emission property is luminescence lifetime. In other embodiments, the emission property is scattering angle. In some embodiments, an increase or decrease in luminescence intensity, lifetime, or scattering angle for one or more scattered wavelengths is related to the presence or absence of an analyte of interest. In some embodiments the detected signal correlates to an analyte that may be an indicator of a biological sample, for example the progress of wound healing or the presence or absence of bacterial infection. In some embodiments, the analyte may be oxygen.

In various embodiments, the levels of light from the light emitting material that are measured by the detectors may be a function of the influence of the analyte on the light emitting material, on the scattering or reflective particles, and/or on the polymer matrix in which the scattering or reflective particles are embedded.

In one embodiment the sensing material includes the light emitting material responsive to an analyte of interest and the plurality of signal enhancing particles are constructed in two or more independently tunable layers, as exemplified in FIGS. 1C-6. In one embodiment, the layers may have a thickness between 10-1000 micrometers, in another embodiment the layers may have a thickness between 100-500 micrometers. In one embodiment each of these independently tunable layers includes the same polymer matrix. In another embodiment these tunable layers include different polymer matrices. In one embodiment the layer embedded with light emitting material is hydrophobic while the layer with the plurality of signal enhancing particles is hydrophilic.

Particles disclosed in this work ideally have a difference in index of reflection from the surrounding polymer matrix. In one embodiment, the difference in index may be no lower than 0.01 and no lower than 1. In some embodiments the particles may include bare metallic, glass-coated metallic, polymeric, or glass particles. In some embodiments the particles are solid, core-shell, or hollow in structure. In some embodiments the particles are glass (e.g. solid or hollow particles such as spheres), titanium dioxide, zinc oxide, barium sulfate, or diamond. Particles may also include polymeric materials such as polystyrene or polyethylene. Other types of particles could be metallic particles including but not limited to gold, silver, tin, and nickel. Additionally, particles can be a combination thereof, such as core-shell particles.

In some embodiments the particles are spherical, faceted, rod, oblate spheroid, or disc shaped. In one embodiment, exemplified in FIG. 8, the particles are rod-shaped and with well-characterized dimensions which can be used as an internal distance calibration registration tool or fiduciary marker for image-based analysis.

In some embodiments the signal enhancing particles have at least one dimension larger than the wavelength of light being scattered or reflected by the particles. In another embodiment, the particles are smaller than 1/10^{th} of the wavelength of light being scattered by them. More specifically, the particles may be no smaller than 0.001 μm (1 nm) and no greater than 100 μm.

In one embodiment, there may be an additional reflecting layer (e.g. a white or mirrored layer) positioned between the sample and the sensing material. In some embodiments the reflecting layer may be a glass microfiber filter paper. In another embodiment the reflecting layer may be white silicone. In one desirable embodiment, there is no reflecting layer obscuring the view of the sample beneath the sensing material. In one such embodiment, the sample is a wound and a clinician can observe the wound through the sensing material.

In one embodiment an oxygen occlusive layer is positioned over the sensing material and sample. In some embodiments the occlusive layer is Tegaderm (about 3,000 cc O₂/m²/day). In other embodiments the occlusive layer may include materials with lower oxygen transmission rates, such as 3M Medical Tape 1524 or 3M Medical Tape 9834. In other embodiments, the occlusive layer 3M medical tape 1513 and 3M medical transfer adhesive.

In one embodiment the polymer matrix provides structure to the sensing material. In some embodiments, the materials may possess an oxygen permeability to ensure rapid exchange of the analyte (e.g., oxygen) but a low permeability for water vapor (e.g., be hydrophobic). Absorbance of water by the matrix may result in structural changes and consequently in changes of the optical properties. In some embodiments the polymer includes at least one of nitrocellulose, agarose, hydrogel, poly(ethylene glycol) (PEG) hydrogel, poly(methyl methacrylate (PMMA), poly(propyl methacrylate) (PPMA), tetraethyl orthosilicate (TEOS), triethoxy(octyl)silane (Octyl-triEOS), 2,4-diethyl-1,5-pentanediol, poly(ethylmethacrylate) (PEMA), Polystyrene (PS), ethylcellulose, Poly(1-trimethylsilyl-1-propyne) (PTMSP), polyethylene terephthalate (PTFE), low-density polyethylene (LDPE), or polydimethyl siloxane (PDMS).

FIG. 1C includes an optional adhesive layer, between the scattering layer and the sample, however in some embodiments the scattering layer itself may include an adhesive, instead of or in addition to the separate adhesive layer. Similarly, in some embodiments such as FIG. 1B the single layer of material may also include adhesive or there may be a separate adhesive layer between the single layer of material and the sample. In some embodiments the polymer matrix may be or include an adhesive. In some embodiments, the adhesive layer may be an acrylate adhesive, a cyanoacrylate adhesive, a urethane or urethane polymer adhesive, an albumin-glutaraldehyde adhesive, or a hydrogel-based adhesive. In some embodiments, the signal enhancing particles may have a different index of refraction than the index of refraction of the adhesive or adhesive-containing layer. In some embodiments the index of refraction signal enhancing particles is different from the index of refraction of the polymer matrix by at least 0.01. In other embodiments, the index of refraction of the signal enhancing particles is different from the adhesive by approximately 0.4.

In some embodiments the sensing material additionally includes one or more reference light emitting materials that are not responsive to the presence of the analyte of interest. In one embodiment, the reference light emitting material is not responsive in emission intensity or spectral properties to the presence of oxygen. In some embodiments the reference dyes may be pH-independent (e.g., display no degradation and no change in optical properties with pH), soluble in the matrix material, possess emission bands that do not overlap with the emission bands of the sensor dye, be excitable at the same wavelength as the sensor dye, and be photostable (e.g., limited photobleaching of the reference dye). In some embodiments the reference dye may be chemically inert, non-toxic, or otherwise not impede or negatively affect wound healing. In one particular embodiment, the reference light emitting material is coumarin. Other reference dyes could be but not limited to fluorescein derivatives, quantum dots, Oregon green, Alexa fluorophores or food dyes.

In some embodiments, the light-emitting material responsive to an analyte of interest is a porphyrin. In one embodiment it is a metalloporphyrin. In certain embodiments it may be a pivaloyl-terminated metalloporphyrin, a silanated porphyrin, or alkynyl porphyrin. Additional materials that could be used as oxygen-sensing light emitting dyes include but not limited to polycyclic aromatic hydrocarbons, such as anthracene, 3,4-benzopyrene, and decacyclene, fullerenes, metal-ligand complexes, such as metal porphyrins, Ru(1,10-phenanthroline)3, Ru(bipyridine)3, and Ir(III) polypyridine complexes, lanthanide complexes, such as europium complexes, Terbium(III) complexes (e.g. Tb(acac)3(phen)), and Eu(tta)3 and Gd(tta)3 complexes.

In other embodiments, the signal enhancing particles may be responsive to a change in the conditions of the sample which may produce a corresponding observable change in the scattering or reflectivity of the particles. In some embodiments the change in scattering or reflectivity may be a result of particles changing size or shape, aggregating with other particles, or disaggregating from a conglomerate as a result of changing sample conditions, producing an increase or decrease in the effective number of particles which in turn affects the amount or properties of the scattered light. One embodiment of this may include particles swelling as a result of exposure to water, resulting in a change in scattering directionality and orientation and a corresponding change in detected luminescence intensity.

For example, without being limited as to theory, Rayleigh scattering dominates when particles are smaller than a tenth of the wavelength of light being scattered. Rayleigh scattering is wavelength dependent and is emitted in all directions. Conversely, Mie scattering dominates when particles are the same size or larger than the wavelength of light being scattered. Mie scattering is less wavelength dependent than Rayleigh scattering, is strongly directional, and is stronger than Rayleigh scattering. In one embodiment, as particles change in the sample conditions, scattering of photons from them may transition from Rayleigh scattering to Mie scattering or from Rayleigh scattering to Mie scattering. This change in dominant scattering mechanism can be leveraged to indicate a change in the sample conditions as a function of particle size. In some embodiments, the changes in sample conditions that may induce changes in particle scattering may include presence of water, release of drug(s), release of exudate or transudate, detection of healthy or diseased tissue, or duration of contact with sample (e.g. to act as a personalized timer for bandage wear time).

In another embodiment, utilizing Mie scattering is exemplified in FIG. 6 wherein a mirrored surface is positioned between the sample and the signal enhancing particles.

In some embodiments, the detector may include one or more of a camera, photodiode and phototransistors, photomultiplier tubes, CCD, CMOS, or a fiber optically coupled detector.

In some embodiments, the excitation source may include a laser, LED, incandescent lamp or flash source (e.g. an electronic flash source) which may be outfitted with filters to permit only a certain wavelength or range of wavelengths to pass. The source may be continuously illuminating or applied to a sample in a single pulse or a series of pulses, or modulated such as via a sine wave or triangle wave. In one embodiment the excitation is applied as a pulse to measure luminescence lifetime. In one embodiment, the excitation source is applied as a pulse to prevent or reduce photobleaching of the light emitting material. In some embodiments, the wavelengths emitted may be ultraviolet, visible, or infrared wavelengths. In some embodiments, the excitation source may include incandescent, fluorescent, laser, LED and gas lamps include mercury and Xenon lamps. In some embodiments, excitation sources may have broad emission bands (covering a broad range of the electromagnetic spectrum) (e. g., incandescent lamps), some may show medium to narrow bands (e.g., LEDs), or some show narrow emission lines (e.g., lasers). In some embodiments, the excitation source may be suitable for single photon excitation or multiphoton excitation. In one embodiment, the excitation source may be a two-photon excitation source. A person skilled in the art will recognize that the choice excitation source depends on the excitation bands of the dyes, excitation power, dimension of the device, and other aspects of the application.

In various embodiments, the excitation source and/or detector may be coupled to a controller, e.g. a computing system, which controls emission of light and collection and processing of data as disclosed herein. The controller may be part of a single device which includes the excitation source and detector or one or more of these may be provided as separate elements. In some embodiments, some or all of the actions of the controller may be distributed among multiple devices that are coupled to one another in a wired or wireless manner, including a networked computer system, a smartphone, a smart watch, or a wearable device (e.g. a performance or athletic monitoring device). Data that is collected and/or analyzed by the controller may be stored on the controller or a related device and/or may be transmitted to another device and/or provided to a subject (e.g. the subject from whom the data is collected) or another party such as a clinician, or may be transferred to an electronic health records system or a fitness app.

In one embodiment the system for measuring the analyte of interest includes exposing the sensing material to conditions where the analyte may be present and monitoring the luminescence intensity, scattering angle, or luminescence lifetime of the light emitting material responsive to the analyte of interest. In one embodiment, the scattering or reflective particles may undergo an interaction with underlying sample conditions, such as absorption of an analyte or release of a drug, that may change the optical scattering or reflective properties of the signal enhancing particles. In some embodiments, the interaction may cause changes in index of refraction, absorptive properties, scattering angle, size, shape, orientation, or number of scatterers. In one embodiment, a computer system may perform the analysis of luminescence intensity, scattering angle, or luminescence lifetime of the light emitting material in order to quantify an increase or decrease in these aforementioned parameters.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Synthesis Methods

To synthesize the luminescent layer, the sensing and reference dyes as well as the matrix polymers are dissolved in an organic solvent, such as but not limited to dichloromethane, ethyl acetate, or dimethylformamide, in the desired ratios to create a casting solution. The solution is cast into a mold or coated on a substrate using but not limited to techniques including spin coating, dip coating, brushing, roll coating, spraying, and flow coating. The scattering particles are added to the casting solution and are deposited directly into matrix material.

To assemble the oxygen-sensing bandage, the dried film is released from the mold or punched out of the substrate and is transferred on the adhesive side of a transparent film dressing, such as but not limited to Tegaderm.

### Example 2: Enhanced-signal Oxygen Sensing

Emission intensity in FIG. 8 was assessed using a Nikon D3400 DSLR camera equipped with 60 mm lens and two bilaterally mounted Vivitar flash units. A blue/UV bandpass filters ( 385/70 nm, Chroma Technologies) was mounted on the lens and red-green dual bandpass filters ( 525/30 nm+ 650/40 nm) were mounted on the flash units. The distance between the lens and the oxygen-sensing film was ~35 cm. Exposure time and aperture number were 0.01 s and f/11, respectively. ISO was set to 100. Afterwards, images were separated in red, green, and blue channels to assess the red and green signal intensity. Luminescence lifetime were measured using the an in-house optical wireless wearable readout system. Further experimental details are provided in Biomedical opt express 2020, 11, 6989-7002, incorporated herein by reference.

1. A sensing system for sensing an analyte, comprising:
a sensing material comprising one or more layers, the sensing material including:
a light emitting material and a plurality of signal enhancing particles disposed within a polymer matrix.

2. The sensing system of claim 1, wherein the light emitting material is sensitive to an analyte.

3. The sensing system of claim 2, wherein the light emitting material undergoes at least one of a change in intensity or a change of emission properties based on exposure to the analyte.

4. The sensing system of claim 2, wherein the light emitting material comprises a metalloporphyrin.

5. The sensing system of claim 2, wherein the analyte comprises oxygen.

6. The sensing system of claim 1, wherein the plurality of signal enhancing particles comprises a plurality of scattering or reflective particles, wherein the plurality of scattering or reflective particles comprises at least one of glass, titanium dioxide, zinc oxide, barium sulfate, diamond, polystyrene, or polypropylene.

7-8. (canceled)

9. The sensing system of claim 2, wherein the sensing material further comprises a reference dye whose emission properties do not undergo at least one of a change in intensity, lifetime, or a change of spectral properties based on exposure to the analyte.

10. The sensing system of claim 1, wherein the sensing material comprises two or more layers,
wherein the light emitting material is disposed within a first layer of the two or more layers and the plurality of signal enhancing particles is disposed within a second layer of the two or more layers, and
wherein the second layer is different from the first layer.

11. The sensing system of claim 10, wherein interaction of the second layer with an analyte causes a change in scattering properties of the second layer, including at least:
wherein interaction of the second layer with the analyte causes a change of index of refraction of the signal enhancing particles of the second layer,
wherein interaction of the second layer with the analyte causes a change of index of refraction of the polymer matrix of the second layer.
wherein interaction of the second layer with the analyte causes a change in scattering angle of light scattered by the signal enhancing particles of the second layer,
wherein interaction of the second layer with the analyte causes a change in shape of the signal enhancing particles of the second layer, or
wherein interaction of the second layer with the analyte causes a change in an effective number of the signal enhancing particles of the second layer.

12-16. (canceled)

17. The sensing system of claim 10, wherein the plurality of signal enhancing particles of the second layer comprises a plurality of Mie scatterers.

18. The sensing system of claim 17, wherein the sensing material further comprises a mirrored layer adjacent to the second layer, wherein the mirrored layer reflects light from the Mie scatterers.

19. The sensing system of claim 1, wherein an index of refraction of the signal enhancing particles is different from an index of refraction of the polymer matrix.

20-21. (canceled)

22. The sensing system of claim 1, wherein the polymer matrix comprises at least one of nitrocellulose, agarose, hydrogel, poly(ethylene glycol) (PEG) hydrogel, Poly(methyl methacrylate (PMMA), poly(propyl methacrylate) (PPMA), tetraethyl orthosilicate (TEOS), triethoxy(octyl)silane (Octyl-triEOS), 2,4-diethyl-1,5-pentanediol, poly(ethylmethacrylate) (PEMA), or polydimethyl siloxane (PDMS).

23. The sensing system of claim 1 ,further comprising an excitation source and a detector,
wherein the excitation source is configured to direct light toward the sensing material, and
wherein the detector is configured to detect light returned from the sensing material.

24. The sensing system of claim 23, wherein the plurality of signal enhancing particles comprises a plurality of rod-shaped particles each having a particular length.

25. The sensing system of claim 24, wherein the detector is configured to collect an image of a rod-shaped particle of the plurality of rod-shaped particles and to determine a size of another structure in the sensing material based on the image of the rod-shaped particle.

26. The sensing system of claim 23, wherein the excitation source comprises at least one of a flash source, a laser, or an LED.

27. The sensing system of claim 23, wherein the excitation source comprises a single photon or multiphoton excitation source.

28. The sensing system of claim 23, wherein the excitation source emits light as at least one of a continuous wave, modulated wave, or one or more discrete pulses.

29. (canceled)

30. The sensing system of claim 1, further comprising an adhesive layer, wherein an index of refraction of the signal enhancing particles is different from an index of refraction of the adhesive layer.

31. (canceled)

32. The sensing system of claim 30, wherein the index of refraction of the signal enhancing particles is different from the index of refraction of the adhesive layer by at least 0.01.

33. The sensing system of claim 30, wherein the index of refraction of the signal enhancing particles is different from the index of refraction of the adhesive layer by approximately 0.4.

34-70. (canceled)
